(19) United States
(12) Patent Application Publication
(10) Pub. No.: US 2015/0072408 A1
(43) Pub. Date: Mar. 12, 2015

(54) BIOLOGICAL REACTOR WITH FULL-WAVELENGTH CONTROLLABLE LIGHT SOURCES

(71) Applicant: SOUTH CHINA BOTANICAL GARDEN, CHINESE ACADEMY OF SCIENCES, Guangdong (CN)

(72) Inventors: Ying Wang, Guangdong (CN); Chunchao Liu, Beijing (CN); Jingzhou Dong, Hubei (CN); Xinlan Xu, Guangdong (CN)

(21) Appl. No.: 14/363,770

(22) PCT Filed: Nov. 10, 2012
(86) PCT No.: PCT/CN2012/084423

§ 371 Date: Nov. 20, 2014

(30) Foreign Application Priority Data
Dec. 8, 2011 (CN) 201110405695.8

(51) Int. Cl. C12M 1/00; C12M 1/34; C12M 1/06

(52) U.S. Cl. CPC C12M 41/06; C12M 27/02; C12M 31/10; C12M 41/26; C12M 41/12; C12M 21/02
USPC 435/292.1

(57) ABSTRACT

A biological reactor with full-wavelength controllable light sources is disclosed. A tank with a top includes a tank cover. A helical agitator of quartz glass inside the tank, and one end of the helical agitator connected with the tank cover. A temperature and pH value sensor inside the tank, and connected with the tank cover. The bottom of the tank is in communication with one end of a fermentation broth outlet pipe which is includes a switch valve, and the other end of the fermentation broth outlet pipe penetrates through the peripheral box. The tank cover includes a medium inlet pipe in communication with the tank, and one end of the medium inlet pipe located out of the tank includes a second sterilizing filter and penetrates through the peripheral box. A side wall of the tank includes a full-wavelength LED device with adjustable wavelength.

### TECHNICAL FIELD

The present invention relates to the field of biological cell photoreactions, and in particularly relates to a biological reactor with full-wavelength controllable light sources, which is applicable to genetic and metabolic researches on light control of wild-type living cells or engineering cells, or industrial light-controlled cell fermentation processes.

### BACKGROUND

A lot of scientific research demonstrates that the vegetative growth and secondary metabolite accumulation of autotrophic biological cells irradiated by a light source with a certain wavelength are orientationally adjusted by the wavelength, and the metabolic pathways of heterotrophic biological cells under the stimulation of a light source with a particular wavelength will be modified, resulting in the accumulation of some biologically active substances. This characteristic of living cells can be used in the industrial production of biologically active substances or medicaments needed by humans. Light sources of different wavelengths each have a certain energy, which can only be absorbed by some particular phytochromes or other photosensitive materials (such as some enzymes and the coenzymes thereof, or non-enzymatic protein receptors) in living cells. Therefore, the gene expression or spatial structure of proteins can be changed by light condition, leading eventually to the corresponding metabolic network variation and enhancing or attenuating the primary growth of living cells, as well as increasing the level of some specific secondary metabolites. This is the theoretical basis for the apparatus according to the present invention. By virtue of this characteristic of living cells, the regulation mechanism of light sources with different wavelengths on metabolic networks can be systematically studied in depth. These unknown photoreaction mechanisms are not only the important contents of scientific research on biological photoreactions but also the core theories in the technical field of cell photoreactions in the cutting-edge biotechnology and bioengineering industries.

Light irradiation of various wavelengths has a certain photochemical effect on the complex metabolic network in a biological cell, and thereby has a regulatory function for signal transduction, gene expression and enzymatic activity. This is of great significance for the bioengineering industry, in which cell culture is adopted for the purpose of producing biologically active substances or medicaments. Therefore, the study of the laws and targets of the effects of light sources of various wavelengths in the full wavelength range on the metabolic network in a living cell is of great theoretical significance for biological cell metabolism photoreactions and is of great practical significance for high-tech development in the bioengineering industry. However, there is so far no scientific apparatus that can fulfill the requirements of the above studies.

### DISCLOSURE OF THE INVENTION

To solve the above-mentioned problems in the prior art, it is an object of the present invention to provide a biological reactor with full-wavelength controllable light sources, which provides full-wavelength light sources in the range of 200 nm to 1000 nm. According to the requirements of scientific researches, a light source controller on the reactor can select light rays of any wavelengths in the wavelength range and combine the same to obtain a mixed or monochromatic light source made up of particular wavelengths combination. Additionally, such light source is utilized for photoreaction researches on fermented cells or plant callus tissues, and in particular suitable for: researches on the use of light irradiation of specific wavelengths for regulation of the expression of some genes, regulation of the accumulation of secondary metabolites, regulation of the growth and differentiation of plant cells, and so on. The object of the present invention can be achieved by the following technical solutions:

A biological reactor with full-wavelength controllable light sources comprises a peripheral box, and a tank made of quartz glass and arranged in the peripheral box. The top of the tank is provided with a tank cover. The reactor further comprises: a glass vent pipe; a helical agitator made of quartz glass and arranged in the tank with one end being connected to the tank cover; and a temperature and pH value sensor, arranged in the tank and one end being connected to the tank cover. The bottom of the tank is in communication with one end of a fermentation broth outlet pipe which is provided with a switch valve, and the other end of the fermentation broth outlet pipe penetrates through the peripheral box. The tank cover is provided with a medium inlet pipe in communication with the tank, and one end of the medium inlet pipe located out of the tank penetrates through the peripheral box and is provided with a second sterilizing filter. A sidewall of the tank is provided with a full-wavelength LED device with an adjustable wavelength, and an outer wall of the peripheral box is provided with a master controller used for adjusting the wavelength of the full-wavelength LED device. One end of the glass vent pipe penetrates through the tank cover and is in communication with the tank, and the other end thereof penetrates through the peripheral box and is provided with a first sterilizing filter.

Said full-wavelength LED device has a wavelength in the range of from 200 nm to 1000 nm, and the regulation of the wavelength of the full-wavelength LED device has a minimum gradient of from 5 nm to 10 nm.

The end of said glass vent pipe that is in communication with the tank extends to the bottom of the tank.

Said glass vent pipes are provided as two pipes.

The upper portion of said tank is provided with a trace sampling opening.

An inner wall of the bottom of said peripheral box is provided with an automatic temperature control heater.

Said full-wavelength LED device comprises a printed board provided with LEDs, the face of the printed board that is provided with LEDs matching up with the periphery of the tank.

Said printed board comprises a first printed board and a second printed board, which are arranged on the two sides of the tank.

The LEDs on said first printed board and the LEDs on said second printed board are distributed in mirror-symmetry mode, and the wavelengths of the LEDs arranged in mirror-image form on said first and second printed boards are distributed in staggered form.

The LEDs on said first printed board and the LEDs on said second printed board are distributed in mirror-symmetry mode, the LEDs on said first printed board are arranged in ascending order of wavelength, and the LEDs on said second printed board are arranged in descending order of wavelength.

The present invention has the following beneficial effects as compared with the prior art:

Simple structure, and easy to operate.

An accurate light source control system is provided to meet the requirements of scientific researches.

Based on the existing fermentation tank technologies, full-wavelength controllable light sources are added to complement the apparatuses and techniques required for researches on biological photoreactions.

The fermentation tank made of quartz glass ensures the transmittance of light sources with various wavelengths, and is useful for real-time observing the growth status of cells or tissues. Moreover, a sealed box is arranged on the periphery of the fermentation tank for heat preservation and light shielding of the fermentation broth, which is therefore prevented from being affected by ambient light.

According to the needs of the fermented cells and the desired products, light sources are changed into a light source panel with a combination of specific wavelengths, which can be used for industrial cell fermentation, and for increasing the rate and amount of cell growth and the yield of the desired products, by virtue of the principle of photoreaction.

### BRIEF DESCRIPTION OF THE DRAWING

FIG. 1 is a structure diagram of a biological reactor with full-wavelength controllable light sources.

In this FIGURE: 1—full-wavelength LED device; 2—helical agitator; 3—glass vent pipe; 4—peripheral box; 5—tank; 6—gas input pipe; 701—first sterilizing filter (which is made of a polystyrene material and has a pore size of from 0.1 to 0.45 microns); 702—second sterilizing filter (which is made of a polystyrene material and has a pore size of from 0.1 to 0.45 microns); 8—medium inlet pipe; 9—temperature and pH value sensor (temperature range: from 0 to 120° C.; pH value sensing range: pH 4.0 to 10.0); 10—automatic temperature control system (which is provided with a heater and a mini-sized quiet compressor, is capable of controlling temperatures in the range of from 0 to 50° C., and is regulated by the master controller); 11—master controller (which consists of a liquid crystal display panel, buttons adapted for selecting light waves and temperatures, and an integrated circuit adapted for regulating the light wave and temperature and displaying the pH value, wherein the liquid crystal panel is useful for displaying the current temperature and setting the temperature, displaying the pH value and the wavelength and setting the same); 12—trace sampling opening; 13—switch valve; 14—tank cover; 15—fermentation broth outlet pipe.

### EMBODIMENTS

### Example 1

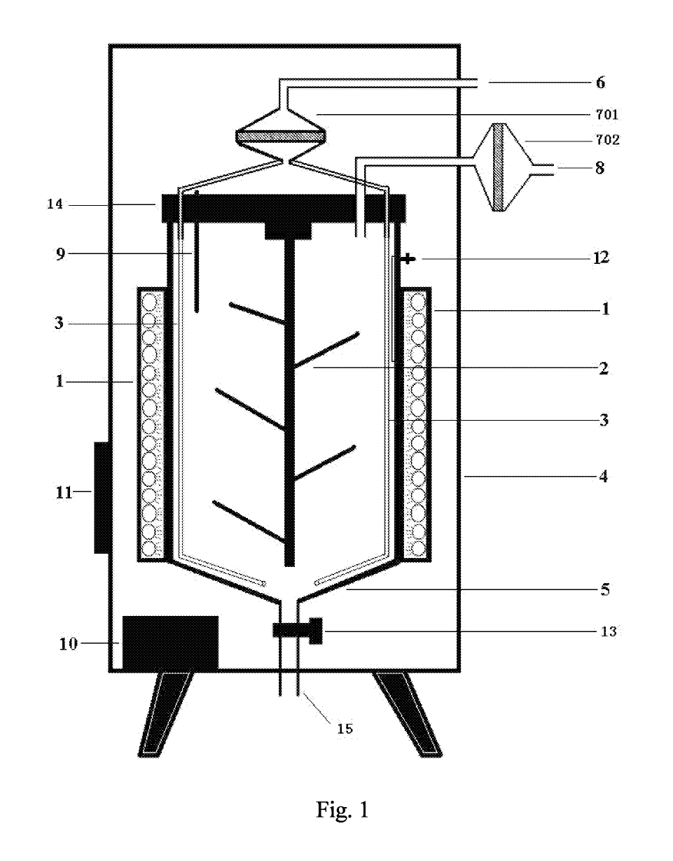

As shown in FIG. 1, a biological reactor with full-wavelength controllable light sources comprises a peripheral box 4, and a tank 5 made of quartz glass and arranged in the peripheral box 4. The top of the tank 5 is provided with a tank cover 14. The reactor further comprises: a glass vent pipe 3; a helical agitator 2 made of quartz glass and arranged in the tank 5 with one end being connected to the tank cover 14; and a temperature and pH value sensor 9 arranged in the tank 5 and one end being connected to the tank cover 14. The bottom of the tank 5 is in communication with one end of a fermentation broth outlet pipe 15 which is provided with a switch valve 13, and the other end of the fermentation broth outlet pipe 15 penetrates through the peripheral box 4. The tank cover 14 is provided with a medium inlet pipe 8 in communication with the tank 5, and one end of the medium inlet pipe 8 located out of the tank 5 penetrates through the peripheral box 4 and is provided with a second sterilizing filter 702. A sidewall of the tank 5 is provided with a full-wavelength LED device 1 with an adjustable wavelength, and an outer wall of the peripheral box 4 is provided with a master controller 11 used for adjusting the wavelength of the full-wavelength LED device 1. One end of the glass vent pipe 3 penetrates through the tank cover 14 and is in communication with the tank 5, and the other end thereof penetrates through the peripheral box 4 and is provided with a first sterilizing filter 701. The full-wavelength LED device 1 has a wavelength in the range of from 200 nm to 1000 nm, and the regulation of the wavelength of the full-wavelength LED device 1 has a minimum gradient of from 5 nm to 10 nm. The end of the glass vent pipe 3 that is in communication with the tank 5 extends to the bottom of the tank 5. There are two said glass vent pipes 3. The upper portion of the tank 5 is provided with a trace sampling opening 12. An inner wall of the bottom of the peripheral box 4 is provided with an automatic temperature control heater 10. The full-wavelength LED device 1 comprises a printed board provided with LEDs, the face of the printed board that is provided with LEDs matching up with the periphery of the tank 5. The printed board comprises a first printed board and a second printed board, which are arranged on the two sides of the tank 5. The LEDs on the first printed board and the LEDs on the second printed board are distributed in mirror-symmetry mode, and the wavelength of the LEDs arranged in mirror-image form on the first and second printed boards are distributed in a staggered form. The LEDs on the first printed board and the LEDs on the second printed board are distributed in mirror-symmetry mode, the LEDs on the first printed board are arranged in ascending order of wavelength, and the LEDs on the second printed board are arranged in descending order of wavelength.

As a preferred embodiment, the LEDs on the first printed board are tightly arranged laterally or longitudinally according to the wavelengths of lamp beads, namely, in the order of 200 nm, 205 nm, 210 nm . . . 1000 nm, 200 nm, 205 nm, 210 nm . . . 1000 nm . . . and so on, until they reach the end of the circuit board; the LEDs on the second printed board are arranged in a direction opposite, in mirror-image form, to the direction in which the LEDs on the first printed board are arranged. Therefore, the fermented cells in the tank are ensured to be homogeneously irradiated by light sources. The control function of the master controller 11 is: a plurality of LED lamp beads of the same wavelength are connected in parallel as a power control unit, and the first and second printed boards are controlled separately.

### Example 2

A laboratory application method of the biological reactor with full-wavelength controllable light sources:

Biomaterial: Rhodiola isolated cells

Medium: Optimized rhodiola cell culture broth

Research on photoreaction conditions:

0-24 Hours after inoculation: The cells were cultured in the dark as the cell adaptation period.

24-124 Hours after inoculation: The cells were irradiated by light with a wavelength of 620 to 630 nm and a surface light intensity of 400 LUX, which were the light wave conditions for the rapid division and growth of cells. Sampling was performed to measure the amount of cell growth and the Rhodioloside content.

124-168 Hours after inoculation: The cells were irradiated by 50% light with a wavelength of 420 to 440 nm+50% light with a wavelength of 620 to 630 nm (“50%” means that a mixed light source consists of 50% light sources of one type and 50% light sources of another type, wherein all the light sources emit light simultaneously; this applies to the cases below), or 40% light with a wavelength of 320 to 350 nm+60% light with a wavelength of 620 to 630 nm, or 10% light with a wavelength of 280 to 320 nm+90% light with a wavelength of 620 to 630 nm, with the surface light intensity of 400 LUX, which were the optimized light wave conditions for the biosynthesis of Rhodioloside. Sampling was performed to measure the Rhodioloside content. The optimal combination of light waves for the biosynthesis of Rhodioloside can be obtained after the repetition of optimization.

### Example 3

A laboratory application method of the biological reactor with full-wavelength controllable light sources:

Biomaterial: Cordyceps militaris strain

Medium: Cordyceps militaris liquid medium

Light control fermentation process:

0-48 Hours after inoculation: The strain was cultured in the dark to adapt themselves and to continue growth.

48-168 Hours after inoculation: The strain was irradiated by light with a wavelength of 620 to 630 nm and a surface light intensity of 400 LUX, which were the optimized light wave conditions for the division and growth of mycelium and for the biosynthesis of adenosine. Then, the maximum mycelial biomass and the maximum yield of adenosine could be achieved.

168-288 Hours after inoculation: The strain was irradiated by light with a wavelength of 420 to 440 nm and a surface light intensity of 400 LUX, which were the optimized light wave conditions for the biosynthesis of cordycepin and carotenoid. Then, the maximum yield of cordycepin and the maximum yield of carotenoid could be achieved.

1. A biological reactor with full-wavelength controllable light sources, comprising:
a peripheral box and a tank made of quartz glass and arranged in the peripheral box, a top of the tank being provided with a tank cover;
a glass vent pipe;
a helical agitator made of quartz glass and arranged in the tank with one end being connected to the tank cover; and
a temperature and pH value sensor arranged in the tank and one end being connected to the tank cover;
wherein a bottom of the tank is in communication with one end of a fermentation broth outlet pipe which is provided with a switch valve, and an other end of the fermentation broth outlet pipe penetrates through the peripheral box, the tank cover is provided with a medium inlet pipe in communication with the tank, and one end of the medium inlet pipe located out of the tank penetrates through the peripheral box and is provided with a second sterilizing filter, a sidewall of the tank is provided with a full-wavelength LED device with an adjustable wavelength, and an outer wall of the peripheral box is provided with a master controller used for adjusting the wavelength of the full-wavelength LED device, one end of the glass vent pipe penetrates through the tank cover and is in communication with the tank, and the other end thereof penetrates through the peripheral box and is provided with a first sterilizing filter.

2. The biological reactor with full-wavelength controllable light sources according to claim 1, wherein the full-wavelength LED device has a wavelength in the range of from 200 nm to 1000 nm, and the regulation of the wavelength of the full-wavelength LED device has a minimum gradient of from 5 nm to 10 nm.

3. The biological reactor with full-wavelength controllable light sources according to claim 2, wherein the end of the glass vent pipe that is in communication with the tank extends to the bottom of the tank.

4. The biological reactor with full-wavelength controllable light sources according to claim 3, wherein said glass vent pipes are provided in two.

5. The biological reactor with full-wavelength controllable light sources according to claim 1, wherein the tank is provided on the upper portion with a trace sampling opening.

6. The biological reactor with full-wavelength controllable light sources according to claim 1, wherein the peripheral box is provided at an inner wall of the bottom with an automatic temperature control heater.

7. The biological reactor with full-wavelength controllable light sources according to claim 1, wherein the full-wavelength LED device comprises a printed board provided with LEDs, the face of the printed board that is provided with LEDs matching up with the periphery of the tank.

8. The biological reactor with full-wavelength controllable light sources according to claim 7, wherein the printed board comprises a first printed board and a second printed board, which are arranged on the two sides of the tank.

9. The biological reactor with full-wavelength controllable light sources according to claim 8, wherein the LEDs on the first printed board and the LEDs on the second printed board are distributed in a mirror-symmetry mode, and the wavelengths of the LEDs arranged in a mirror-image form on the first and second printed boards are distributed in a staggered form.

10. The biological reactor with full-wavelength controllable light sources according to claim 8, wherein the LEDs on the first printed board and the LEDs on the second printed board are distributed in a mirror-symmetry mode, the LEDs on the first printed board are arranged in ascending order of wavelength, and the LEDs on the second printed board are arranged in descending order of wavelength.
